(19) United States
(12) Patent Application Publication
(10) Pub. No.: US 2005/0043610 A1
(43) Pub. Date: Feb. 24, 2005

(54) Device for adaptation of in particular surgical instruments such as pointing devices

(76) Inventors: Udo Warschewske, Berlin (DE); Karl-Heinz Reinwald, Berlin (DE)

Correspondence Address: WOOD, HERRON & EVANS, LLP, 2700 CAREW TOWER, 441 VINE STREET, CINCINNATI, OH, 45202, US

(21) Appl. No.: 10/493,005

(22) PCT Filed: Oct. 17, 2002
(86) PCT No.: PCT/EP02/11642

(30) Foreign Application Priority Data
Oct. 18, 2001 (DE) 10151398.4

(51) Int. Cl. A61B 5/05

(52) U.S. Cl. 600/409

(57) ABSTRACT

The invention relates to a device for the adaptation of in particular surgical instruments, such as pointing devices. Said adaptation device comprises a magnetic field sensor or the like, a receiver device for a surgical instrument and a locking option for said surgical instrument. Said receiver device, which is associated with the respective surgical instrument and exclusively adapted thereto, permits a precise positioning of said instrument in relation to the sensor by means of a locking device. Said locking device as well as the receiver device design prevent an unintentional loosening or a displacement of the surgical instrument in the receiver device. A time consuming recalibration of said magnetic field senor on assembling and disassembling the surgical instrument is thus avoided.

The present invention relates to a device for the adaptation of in particular surgical instruments as pointing devices in a neuro-navigation system or a comparable system for monitoring surgical interventions, according to the precharacterising part of claim 1.

Operative interventions that take place in a region of the body that is of complicated anatomical structure and that accordingly places stringent demands on the accuracy of the operative procedures are increasingly carried out using navigation systems that assist the operator (surgeon), in which the operator acts with the assistance of additional diagnostic techniques that show them the exact anatomical position of the diseased region of the body. For this purpose a detailed image of the future field of operation and in particular of the diseased region of the body to be removed or treated is built up by means of a method, such as for example nuclear spin tomography or computer tomography, that provides 2D diagnostic representations, and is stored in an image databank.

During the operation a tracking device serves to detect the instantaneous position of a sensor scanning the field of operation, to display this to the operator on a monitor and to compare this with the data obtained from the established image databank. In this way it is possible to localise unequivocally and accurately the area to be treated. This device, termed inter alia a neuro-navigation system, is used in particular in brain surgery, belongs to the prior art, and is known from PCT WO96 08 209. In this connection the tracking device is designed as a system comprising a magnetic field transmitter and magnetic field sensor that establishes within the navigation area the position of the sensor by measuring the magnetic field strength.

Various embodiments and arrangements of in particular the magnetic field sensor are known from the prior art. Thus, for example, the sensor can be incorporated in a special pointing instrument that enables the field of operation to be scanned with the sensor, in which the operator directly contacts the anatomical structures of the patient. A replacement of the pointing instrument is in this case automatically recognised by the navigation system, wherein the special calibration data for the exact calculation of the position of the measurement tip of the pointing instrument are stored in the system and are thus immediately taken into account. A calibration of the instruments before the start of or during the surgical intervention is omitted in this case.

The solutions according to the prior art do not however enable the surgical instrument used in each case to be positioned in a controlled manner by means of the navigation, since sensors and pointers are manipulated separately from the surgical instrument and are not carried on the said instrument.

It would be advantageous to be able to combine the sensor with the instrument in a suitable manner and to be able to use the instrument itself as a pointing instrument. The solution of such an object must enable the sensor to be secured to the surgical instrument in as simple and inexpensive a way as possible, and the securement itself must not complicate or hinder the manipulation of the instrument and in addition must be stable and secure against unintentional loosening as well as positional displacements of the sensor in relation to the tip of the instrument and must also permit a rapid and complication-free exchange of the instrument. In particular, the last point of the outlined problem presents some difficulties since the sought solution should permit an automatic loading of the calibration data in order to avoid a complicated calibration of the instruments before or during the operation. The securement of the sensor to the surgical instrument must therefore be sensitive to the type of instrument itself.

The solution of the aforementioned object of the invention is achieved with a device according to the features of claim 1, the subclaims disclosing at least convenient modifications and improvements.

The securement of the magnetic field sensor is carried out according to the invention by a receiver device which, by means of predominantly interlocking-type connections between the magnetic field sensor and surgical instrument, solves all the aforementioned problems as regards the requisite unique connection as well as the necessary stability and also the rapid replaceability.

The device consists of an adaptor attachment that is secured to the surgical instrument, a receiver device for the magnetic field sensor and arrangements for a stable connection between the adaptor attachment and magnetic field sensor receiver.

The surgical instrument provided with the adaptor attachment is inserted into an adaptor receiver device provided for this purpose in the magnetic field sensor receiver. The adaptor attachment and correspondingly thereto the adaptor receiver device are designed so that a rotation of the surgical instrument in the adaptor receiver device is not possible. Conveniently this is accomplished by means of a square-shaped section or a section of comparable shape.

The unambiguous association of the magnetic field sensor receiver with the corresponding surgical instrument is accomplished by means of studs arranged substantially longitudinally on the for example square-shaped portion of the adaptor attachment, as well as by depressions incorporated in a similar manner thereto in the adaptor attachment receiver. Depending on the specific type of surgical instrument that is connected to the magnetic field sensor corresponding to it, the width and shape of the studs on the square-shaped portion of the adaptor attachment and on the depressions corresponding thereto in the adaptor attachment receiver are designed so that the corresponding surgical instrument, such as a lock, matches only the magnetic field sensor receiver associated with it and accordingly possible mistakes are prevented.

The exact positioning of the instrument in the longitudinal direction is ensured by virtue of the fact that the adaptor attachment receiver is provided with a stop means and cooperates with the locking device between the magnetic field sensor receiver and adaptor attachment in such a way that a locking of the connection is possible only if the surgical instrument together with its adaptor attachment is inserted as far as the stop means in the adaptor attachment receiver.

A shape incorporated in the adaptor attachment, conveniently a depression in the form of a groove, serves to lock the connection. A profiled cylinder flattened at a point on its cylindrical surface is guided over a bore made in the magnetic field sensor receiver in such a way that its rounded portion engages in an interlocking-type manner in the groove of the adaptor attachment when the locking device is closed. The profiled cylinder is hereby rotated by means of a locking lever into the corresponding position.

The locking lever itself is prevented from executing an unintentional rotation from its locking position. A contour on the locking lever serves for this purpose, which conveniently engages in the locking position in an interlocking-type manner with the counterpiece corresponding to it on the outer surface of the magnetic field sensor receiver.

The position of the groove on the adaptor attachment is dimensioned so that the rounded portion of the profiled cylinder of the locking device can only rotate in the groove if the adaptor attachment is inserted as far as the stop means in the adaptor attachment receiver. The device can thus only be locked if the correct surgical instrument is correctly connected to the magnetic field sensor receiver.

Conveniently further modifications of the aforedescribed device may be implemented, such as for example a probe and similar manipulating elements for operating and for switching off the magnetic field sensor and controlling its functioning.

The invention will now be described in more detail hereinafter with reference to an example of implementation and to the accompanying drawings, in which:

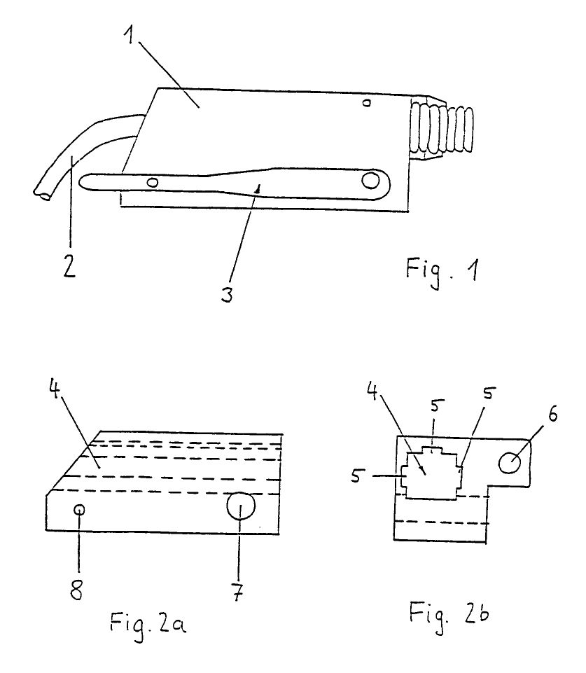

FIG. 1 is an overall view of the device comprising a magnetic field sensor receiver in conjunction with the surgical instrument, in this case a surgical suction device;

FIG. 2a is a front view of the magnetic field sensor receiver;

FIG. 2b is a side view (viewed by the operator) of the magnetic field sensor receiver;

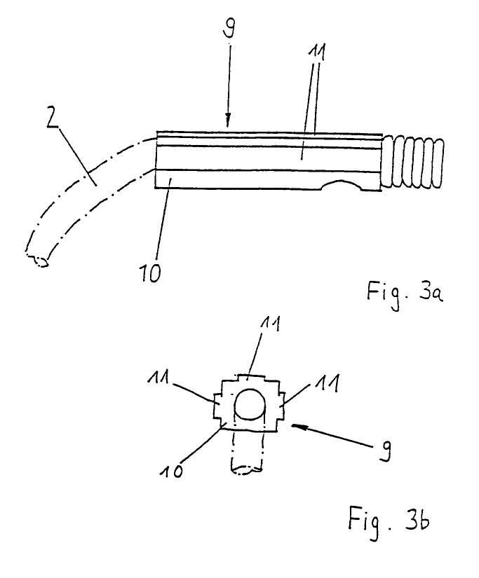

FIGS. 3a, b show the adaptor attachment on the surgical suction device, and

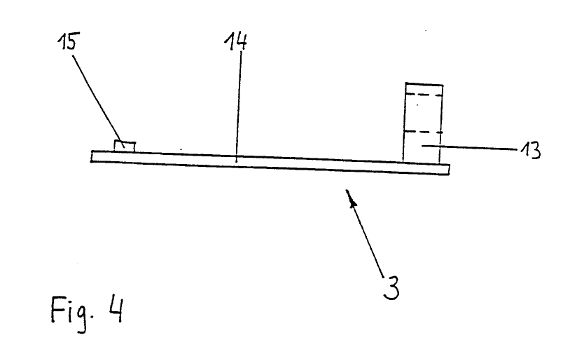

FIG. 4 shows the locking device with locking lever and profiled cylinder.

The device according to the example of implementation consists of the magnetic field sensor receiver 1, which surrounds the surgical suction device 2. The magnetic field sensor receiver 1 accommodates the locking device 3 as well as the adaptor attachment 9, shown in more detail in FIGS. 3a, b. The magnetic field sensor receiver 1 includes in its interior a continuous clear square-shaped profile 4 with depressions 5, as well as a recess 6 for receiving the magnetic field sensor. In addition the magnetic field sensor receiver 1 includes a bore 7 for receiving the profiled cylinder of the locking device 3. A further recess in the form of a blind bore 8 is furthermore provided that serves to lock the closure device 3.

The adaptor attachment 9 is mounted in a frictional manner on the surgical suction device 2. The attachment substantially consists of a square-shaped section 10 that is provided with protuberances in the form of studs 11 extending in the longitudinal direction of the profiled section. The studs 11 are dimensioned so that the adaptor attachment 9 can be inserted into the clear square-shaped or polygonal section 4 of the magnetic sensor receiver 1 only if these fit in an interlocking-type manner in the depressions 5. Since the studs 11 and the depressions 5 are designed so that their dimensions unambiguously match a specific surgical instrument, this ensures that only the surgical instrument 2 with the adaptor attachment 9 corresponding to it can be inserted into the associated magnetic sensor receiver 1, incorrect matchings thereby being avoided.

A secure locking of the surgical instrument 2 is achieved on the one hand by the square-shaped profiled section 10, whereby a rotation of the surgical instrument 2 in the magnetic sensor receiver 1 is avoided. In addition the clear square-shaped section 4 has at its end facing the tip of the instrument a stop means that prevents the adaptor attachment 9 being inserted too far. Together with the groove 12 and the profiled cylinder 13 of the locking device 3, the adaptor attachment 9 in its longitudinal direction is fixed unambiguously in its position in the square-shaped or polygonal section 4 of the magnetic sensor receiver 1 as soon as the locking device 3 is closed.

The locking device 3 consists of the profiled cylinder 13, the locking lever 14 and a knob 15. The profiled cylinder 13 is flattened on one side of its cylindrical surface so that this flattened portion can abut flush with the surface of the clear square-shaped portion 4 when the locking device 3 is open. It is then possible to insert the adaptor attachment as far as the stop means. In this case the groove 12 abuts flush against the curved portion of the bore 7 and the profiled cylinder together with its curved cylindrical part can be rotated by means of the locking lever 14 into the groove 12, so that it is no longer possible for the adaptor attachment 9 to slide in the longitudinal direction.

The closure lever 14 can be locked on the outer surface of the magnetic sensor receiver 1 as soon as the closed state is adopted. For this, the knob 15 engages in the blind bore 8 located on the outer surface of the magnetic sensor receiver 1 and is held in this position by the resilient clamping of the locking lever 14.

As a further modification of the aforedescribed device it is possible for example to provide a probe that is integrated in the sensor receiver and by means of which the manipulation of the magnetic sensor can be adapted to the respective requirements of the operation procedure.

Further modifications are disclosed in the subclaims. The following reference numeral list serves to illustrate the accompanying figures. The same reference numerals are used for identical or equivalent parts.

Reference Numeral List:

1 Magnetic sensor receiver
2 Surgical instrument (surgical suction device)
3 Closure device
4 Square-shaped or polygonal section
5 Depressions
6 Recess for receiving the magnetic field sensor
7 Bore
8 Blind bore
9 Adaptor attachment
10 Square-shaped section
11 Studs
12 Groove
13 Profiled cylinder
14 Closure lever
15 Knobs

1-18. (Canceled).

19. An apparatus for the adaptation of a surgical instrument as a pointing device in a neuro-navigation system or a comparable system for monitoring surgical interventions, the apparatus comprising:
a sensor receiver;
an adaptor attachment adapted for use with the surgical instrument, and a positioning of the surgical instrument with respect to the sensor receiver being ensured by the adaptor attachment; and
a securement device operable to lock the adaptor attachment with respect to the sensor receiver to prevent, during the use of the surgical instrument, an unintentional loosening and/or a spatial displacement of the surgical instrument with respect to the sensor receiver, and a rapid and simple installation and removal of the surgical instrument from the sensor receiver is provided thus avoiding a calibration of the sensor receiver.

20. The apparatus of claim 19 wherein the adaptor attachment is uniquely designed for, and unambiguously associated with, the surgical instrument.

21. The apparatus of claim 20 wherein the adaptor attachment comprises a polygonal attachment.

22. The apparatus of claim 20 wherein the adaptor attachment is connected in a frictional relationship with the sensor receiver.

23. The apparatus of claim 20 wherein the adaptor attachment comprises a groove on its lower side.

24. The apparatus of claim 23 wherein the adaptor attachment comprises a profiled structure present on at least one surface and extending substantially in a longitudinal direction of the adaptor attachment.

25. The apparatus of claim 24 wherein the shape of the profiled structure identifies, and is uniquely associated with, a type of the surgical instrument.

26. The apparatus of claim 25 wherein the sensor receiver comprises a magnetic field sensor with a receiver.

27. The apparatus of claim 26 wherein the receiver comprises a clear inner guide providing an interlocking-type reception for the adaptor attachment.

28. The apparatus of claim 27 wherein the inner guide comprises a polygonal opening with a stop that fits the adaptor attachment in an interlocking-type manner.

29. The apparatus of claim 28 wherein the polygonal section of the adaptor attachment comprises a depression structure comprising depressions running in the longitudinal direction.

30. The apparatus of claim 29 wherein the depression structure of the adaptor attachment provides an unambiguous matching with the polygonal opening of the sensor receiver and thus with the type of the surgical instrument.

31. The apparatus of claim 30 wherein the profiled structure on the polygonal section of the adaptor attachment fits in an interlocking-type manner in the polygonal opening in the sensor receiver.

32. The apparatus of claim 19 wherein the securement device comprises a locking lever and a profiled cylinder between the sensor receiver and the adaptor attachment.

33. The apparatus of claim 32 wherein the profiled cylinder comprises at least a semi-circular cam having a locked state engaging in an interlocking-type manner a groove on the adaptor attachment.

34. The apparatus of claim 32 wherein the profiled cylinder is connected to one end of the knob and is insertable into the sensor receiver to engage the adaptor attachment in an interlocking-type manner in a depression provided on an outer surface of the adaptor attachment.

35. The apparatus of claim 34 wherein the sensor receiver comprises a stop limiting insertion of the adaptor attachment into the sensor receiver, so that an actuation of the securement device is possible only when the adaptor attachment is inserted as far as the stop in the sensor receiver.

36. The apparatus of claim 35 wherein the locking lever further comprises a lock engageable with the sensor receiver to limit motion of the locking lever with respect to the sensor receiver.
